(19) United States
(12) Patent Application Publication
(10) Pub. No.: US 2016/0120507 A1
(43) Pub. Date: May 5, 2016

(54) ULTRASOUND DIAGNOSTIC DEVICE

(71) Applicant: HITACHI ALOKA MEDICAL, LTD., Tokyo (JP)

(72) Inventors: Atsushi NINOMIYA, Tokyo (JP); Katsumi USAMI, Tokyo (JP); Kazuyuki YANASE, Tokyo (JP); Masaru YOKOYAMA, Tokyo (JP)

(21) Appl. No.: 14/892,693

(22) PCT Filed: May 28, 2014
(86) PCT No.: PCT/JP2014/064144

§ 371 Date: Nov. 20, 2015

(30) Foreign Application Priority Data
May 31, 2013 (JP) 2013-115269

(51) Int. Cl. A61B 8/00; G06F 3/147; G06F 1/16; G06F 3/14

(52) U.S. Cl. CPC A61B 8/464; A61B 8/56; A61B 8/462; G06F 3/1431; G06F 3/147; G06F 1/1649

(57) ABSTRACT

Provided is a compact ultrasound diagnostic device having a high degree of freedom in a viewing position and a viewing angle of a display panel and having excellent operability. The ultrasonic diagnostic device includes a main body (10) that includes an operation panel (20); a first display (30) connected to the main body through a supporter (60); and a second display (40) detachably connected to the main body. The second display (40) includes a transmission/reception circuit that performs transmission or reception with the main body (10). When the sub display 40 is detached, a connection unit (25) on a side of the main body, to which the sub display 40 is connected, functions as a connection unit of the main display 30.

### TECHNICAL FIELD

The present invention relates to an ultrasound diagnostic device, and more particularly, to an ultrasound diagnostic device including two displays.

### BACKGROUND ART

In an ultrasound diagnostic device, in order to allow a patient and other persons in addition to an examiner to easily view a display panel that displays an ultrasound image, various types of mechanisms that support the display panel to a device main body to rotate (turn) or move up and down the display panel with respect to the device main body have been proposed (PTL 1). Further, most of the ultrasound diagnostic devices in the related art include a mechanism that folds the display panel from a standing posture to a horizontal posture when the device is not used, for example.

Further, in order to increase the degree of freedom in viewing the display panel, a device in which a display unit provided with a display panel is provided independently of a main body of an ultrasound diagnostic device and a cart portion of the display unit is connectable to a cart portion of the main body has been proposed (PTL 2).

### CITATION LIST

### Patent Literature

PTL 1: JP-A-2011-147786

PTL 2: JP-A-2010-46374

### SUMMARY OF INVENTION

In an ultrasound diagnostic device in the related art in which a display panel is fixed to a device main body, a rotating mechanism or a vertical movement mechanism is provided as a support mechanism, but there is a restriction in a movement range of the display panel. On the other hand, when a display panel is mounted as a display unit structure provided separately from a main body, the entire device including the main body and the display unit becomes bulky, and a wide space is necessary for transportation or accommodation.

An object of the invention is to provide an ultrasound diagnostic device which is generally compact, having a high degree of freedom in a viewing position and a viewing angle of a display panel and having excellent operability.

In order to achieve the above object, an ultrasound diagnostic device of the invention includes two displays. One display (first display) is fixed to a device main body through a support mechanism, and the other display (second display) is detachably mounted to the main body. Here, the “main body” of the invention represents a device main body including an operation panel, and “detachably mounted to the main body”, “connected to the main body”, or the like includes a case of “detachably mounted to the operation panel” or “connected to the operation panel”.

A display connection unit for connection of at least one of two displays is provided in the main body (operation panel). The ultrasound diagnostic device is operated in a dual-display mode where two displays are operated when the detachable second display is connected to the display connection unit. The first display is supported to the main body to be movable from a rear position of the second display to an upper or front position thereof, and can move without collision with the second display connected to the main body.

When the detachable second display is detached from the display connection unit, the first display is connected to the display connection unit, and the ultrasound diagnostic device is operated in a mono-display mode where an ultrasound image or the like is displayed on one display. Here, the first display is connected to the display connection unit. Further, the second display detached from the display connection unit performs transmission or reception with the main body in a wireless manner to function as a display panel.

According to the invention, since the second display which is detachably mounted to the main body, in addition to the first display fixed to the main body, is provided, it is possible to view an ultrasound image at a free posture by the second display detached from the main body. Further, providing a connector for the first display and a connector for the second display in the main body as a common one connection unit, and connecting the second display to the main body, it is possible to increase the degree of freedom in display such as a large screen display or a division display using the two display panels.

Further, by providing a mechanism that allows an operation of jumping over the second display in a support mechanism of the first display, even in a state where the second display is connected to the main body, it is possible to smoothly perform the operation of the first display.

### BRIEF DESCRIPTION OF DRAWINGS

FIG. 1 is a perspective view illustrating an overall outline of an ultrasound diagnostic device according to a first embodiment.

FIG. 2 is a side view illustrating main parts of the ultrasound diagnostic device shown in FIG. 1.

FIG. 3 is a diagram illustrating a second display (sub display) in a state of being detached from a main body.

FIG. 4(a) is a perspective view illustrating a display connection unit of the main body, and FIG. 4 (b) is a perspective view illustrating an end portion of the display connected to the connection unit.

FIG. 5 is a side sectional view illustrating the connection unit to which a display is connected.

FIG. 6 is a diagram schematically illustrating a support mechanism for an operation panel.

FIG. 7 is a diagram illustrating a supporter of a first display (main display) supported by the main body.

FIG. 8 is a diagram illustrating a posture example (posture A) of the second display connected to the connection unit.

FIGS. 9(a) and 9(b) are diagrams illustrating a positional change of the first display at the posture A of the second display shown in FIG. 8.

FIG. 10 is a diagram illustrating another posture example (posture B) of the second display connected to the connection unit.

FIG. 11 is a diagram illustrating a positional change of the first display at the posture B of the second display shown in FIG. 10.

FIGS. 12(a) and 12(b) are diagrams illustrating an operation when the first display supported by the main body is connected to the connection unit.

FIG. 13 is a perspective view illustrating a display supporter of an ultrasound diagnostic device according to a second embodiment.

FIG. 14 is a side view illustrating a dual-display state in the second embodiment.

FIG. 15 is a side view illustrating a mono-display state in the second embodiment.

FIGS. 16(a) and 16(b) are diagrams illustrating a motion of a first display in the ultrasound diagnostic device of the second embodiment.

FIG. 17 is a perspective view illustrating a modification example of the display supporter of the second embodiment.

FIG. 18 is a side view illustrating a dual-display state in the modification example shown in FIG. 17.

FIG. 19 is a side view illustrating a mono-display state in the modification example shown in FIG. 17.

FIG. 20 is a sectional view illustrating main parts of an ultrasound diagnostic device according to a third embodiment.

FIG. 21 is a diagram illustrating a transverse movement of a display of an ultrasound diagnostic device of the third embodiment.

FIG. 22 is a functional block diagram illustrating an ultrasound diagnostic device according to a fourth embodiment.

FIG. 23 is a flowchart illustrating an operation of the ultrasound diagnostic device of the fourth embodiment.

FIG. 24 is a diagram illustrating a display screen example of a second display.

### DESCRIPTION OF EMBODIMENTS

An ultrasound diagnostic device of embodiments of the invention includes a main body that includes an operation panel, a first display that is connected to the main body through a supporter, and a second display that is detachably connected to the main body. The second display that is detachably connected to the main body includes a transmission/reception circuit that performs transmission or reception of a signal with the main body. Further, the main body includes a display connection unit that is detachably connected to the second display, and the first display and the second display include an end portion that is engaged with the display connection unit. The ultrasound diagnostic device of the embodiments may be operated in an operating mode (dual-display mode) where the second display is connected to the display connection unit and the first display and the second display function as displays connected to the main body, and in an operating mode (mono-display mode) where the second display is detached from the main body and the first display is connected to the display connection unit.

Hereinafter, the embodiments of the ultrasound diagnostic device of the invention will be described with reference to the accompanying drawings.

### First Embodiment

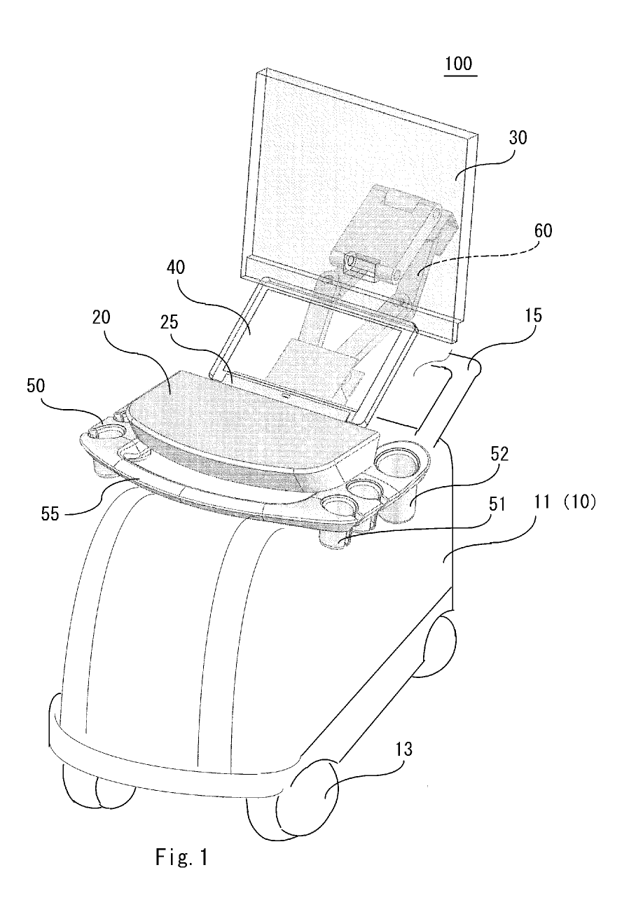

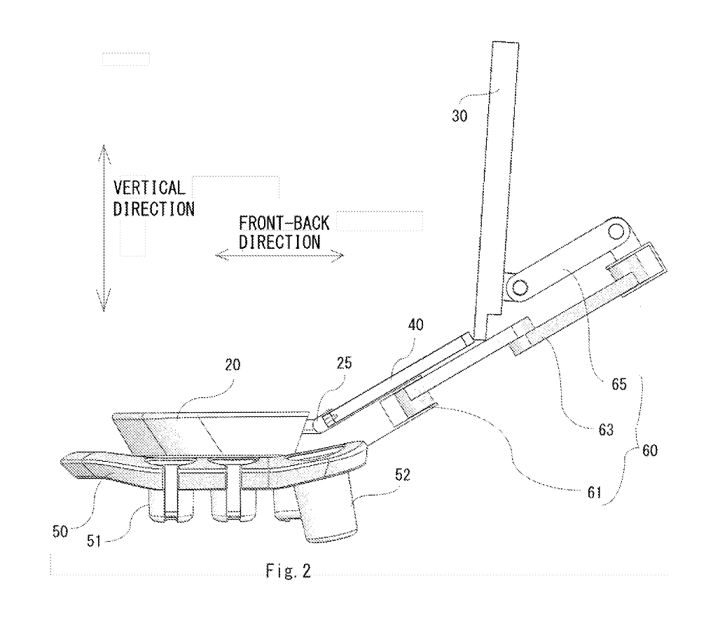

FIG. 1 is a perspective view illustrating an overall outline of an ultrasound diagnostic device 100 of a first embodiment, and FIG. 2 is a side view illustrating main parts of the ultrasound diagnostic device shown in FIG. 1. In the figures, a left side is a front side of the device, and a side opposite thereto is a rear side. As shown in the figures, the ultrasound diagnostic device 100 includes a main body 10, an operation panel 20 fixed to the main body 10 through a support mechanism, two displays 30 and 40, and a probe housing 50 for storing an ultrasound probe (not shown), as main components. The displays 30 and 40 have respectively independent functions and thus are not in a main-sub relationship, but hereinafter, for ease of description, the display (first display) 30 is referred to as a main display 30, the display (second display) 40 is referred to as a sub display 40. Further, when it is not necessary that the displays 30 and 40 are distinguished from each other, the displays 30 and 40 are simply referred to as displays.

The main body 10 includes a housing 11 that accommodates therein an electronic circuit and the like which are main components of the ultrasound diagnostic device. Further, a cart 13 is provided in a lower portion of the housing 11, and a handle 15 used when an operator moves the device is provided in an upper portion of the housing 11.

The operation panel 20 is a component that performs an input operation for operating the main body 10. Although not shown, input devices (not shown) such as buttons, keys, or a pointing device for receiving an input are provided on an upper surface of the operation panel 20 at a predetermined arrangement. The operation panel 20 is supported by the support mechanism fixed to the housing 11 and is disposed in an upper portion of the housing 11. The support mechanism for the operation panel 20 includes a mechanism capable of rotating the operation panel 20 around a vertical axis, moving the operation panel 20 in the horizontal direction (front-back direction/lateral direction), swing, and moving the operation panel 20 in the vertical direction. Further, on the rear side of the operation panel 20, a display connection unit (hereinafter, simply referred to as a connection unit) 25 for connection of the main display 30 and the sub display 40 is provided. Details of the structure of the connection unit 25 will be described later.

The main display 30 is supported by a supporter fixed to an upper portion of the housing 11 or the rear surface of the operation panel 20, and is electrically connected to the main body 10 through a cable (not shown). In this embodiment, although its configuration is not particularly limited, the main display 30 has a display surface of a screen larger than that of the sub display 40, and shares display content with the sub display 40 according to characteristics such as the number of pixels or definition in a state where the sub display 40 is connected to the main body 10.

The supporter of the main display 30 includes a connecting mechanism 60 which is a combination of plural mechanisms capable of rotating the main display 30 around a vertical axis, moving the main display 30 in the horizontal direction, and moving the main display 30 in the vertical direction. Although details of the connecting mechanism 60 will be described, the main display 30 can move from a rear surface (rear side) position to a front surface position with reference to the sub display 40 connected to the connection unit 25 by the connecting mechanism 60. Further, a structure connected to the connection unit 25 provided in the operation panel 20 is provided at a lower end of the main display 30, and in a state where the sub display 40 is detached from the connection unit 25, the main display 30 may be connected to the connection unit 25 through the structure.

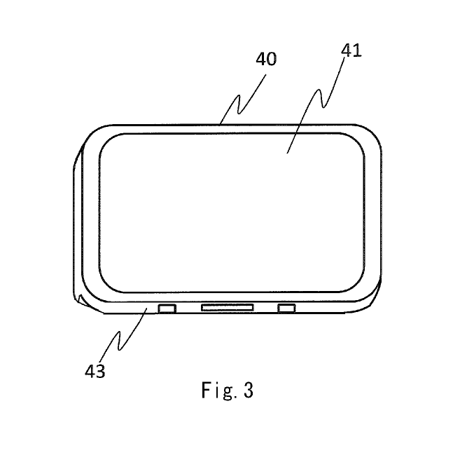

As shown in FIG. 3, the sub display 40 is a tablet-type display device, and includes a display surface 41 of a touch panel type. A drive circuit and a transmission/reception circuit for performing transmission or reception of a signal with the main body 10, or the like are stored in the sub display 40. Further, a structure connected to the connection unit 25 provided in the operation panel 20 is provided on one surface 43 orthogonal to the display surface 41. The sub display 40 is detachably mounted to the connection unit 25, and is electrically connected to the main body 10 through a terminal provided in the connection unit 25 in a state of being connected to the connection unit 25, so that transmission or reception of a signal or data can be performed with the main body 10. In a state where the sub display 40 is detached from the connection unit 25, the sub display 40 functions as an independent tablet-type display device. In addition, the sub display 40 may perform the transmission or reception of the signal or data with the main body 10 in a wireless manner, and in this case, the sub display 40 functions as a simple ultrasound diagnostic device. The function of the sub display 40 that functions as the simple ultrasound diagnostic device will be described in other embodiments.

The probe housing 50 is a member including a probe holder 51 and a jelly holder 52 for accommodating an ultrasound probe or jelly. The probe housing 50 is fixed to an upper end of the support mechanism of the operation panel 20 so that the probe holder and the jelly holder are disposed on opposite sides of the operation panel 20, and may move integrally together with the operation panel 20. A portion that connects right and left portions of the probe housing 50 on the front side of the operation panel 20 also serves as an operation handle 55 of the operation panel 20. By operating the operation handle 55, the operation panel 20 can rotate or slide, for example.

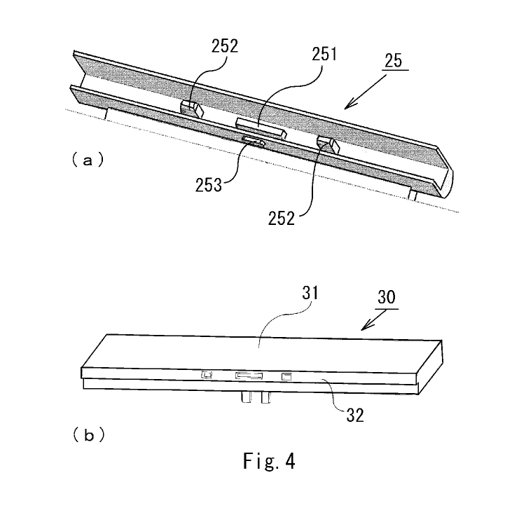

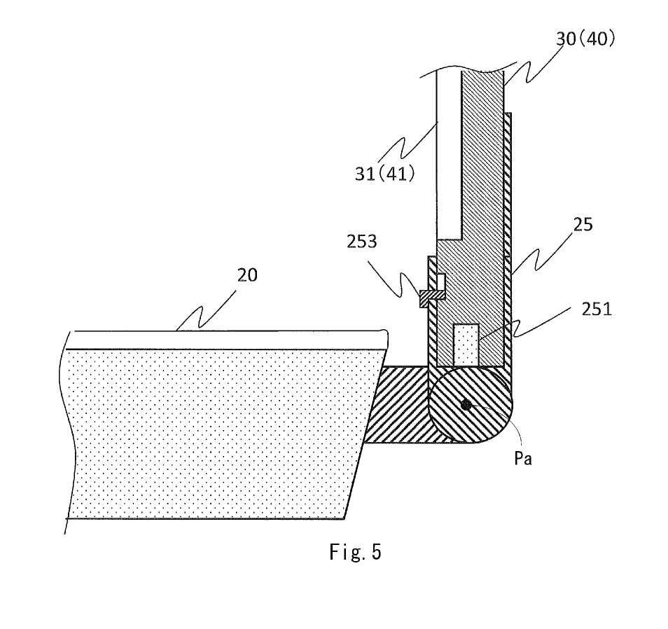

Next, the structure of the connection unit 25 of the operation panel 20 and the structure of the end portion of the display 30 (40) connected thereto will be described with reference to FIGS. 4 and 5. FIG. 4(a) is a top view of the connection unit, FIG. 4(b) is a diagram illustrating an end portion of a display connected to the connection unit, and FIG. 5 is partial sectional view illustrating a state where the display is connected to the connection unit.

The connection unit 25 is provided to be rotatable around an axis Pa along one surface of a housing that forms the operation panel 20, and thus, an angle of the display panel connected to the connection unit 25 with respect to the operation panel 20 can be changed. As shown in FIGS. 4(a) and 5, the connection unit 25 is an elongated member formed by a concave portion having a U-shaped (channel shape) section, and is mounted to the rear surface of the operation panel 20 so that its length direction is parallel to the rear surface of the operation panel 20. The concave portion serves as an accommodating part that accommodates an end portion of the display 30 or 40, and there are provided on the bottom thereof, a connector 251 for electrically connecting the display 30 or 40 to the main body 10 and a protrusion 252 which serves as a guide when the display panel is inserted into the accommodating part. Further, a locking mechanism 253 that fixes the display at a position where the display panel is inserted into the concave portion is provided. As the locking mechanism 253, a known mechanism in which a key-shaped member slides or rotates to be engaged with a hole of an opponent to be locked is used.

On the other hand, as shown in FIG. 4(b), in one end surface of the display 30 or 40, a portion having approximately the same width as an inner width of the concave portion of the connection unit 25 is formed. Further, in an end surface of this portion, holes with which two protrusions 252 of the connection unit 25 are engaged are formed at the same interval as that of the protrusions 252. A concave terminal to be connected to the connector 251 is formed at an intermediate position between the two holes. With such an end structure, when the end portion of the display 30 or 40 is inserted into the concave portion of the connection unit 25, the display 30 or 40 is mechanically and electrically connected to the main body. Since the main display 30 is connected to the main body through the cable, a concave portion that accommodates the connector 251 may simply function as a mechanical connector. In this case, the main body may recognize whether the display connected to the connection unit 25 is the main display 30 or the sub display 40 based on the connection of the sub display 40 to the connector 251.

The main body of the ultrasound diagnostic device in the invention refers to an entire device including the main body 10 and the operation panel 20 excluding the displays 30 and 40.

Next, details and operations of the respective support mechanisms of the operation panel 20 and the main display 30 in the ultrasound diagnostic device 100 of this embodiment will be described.

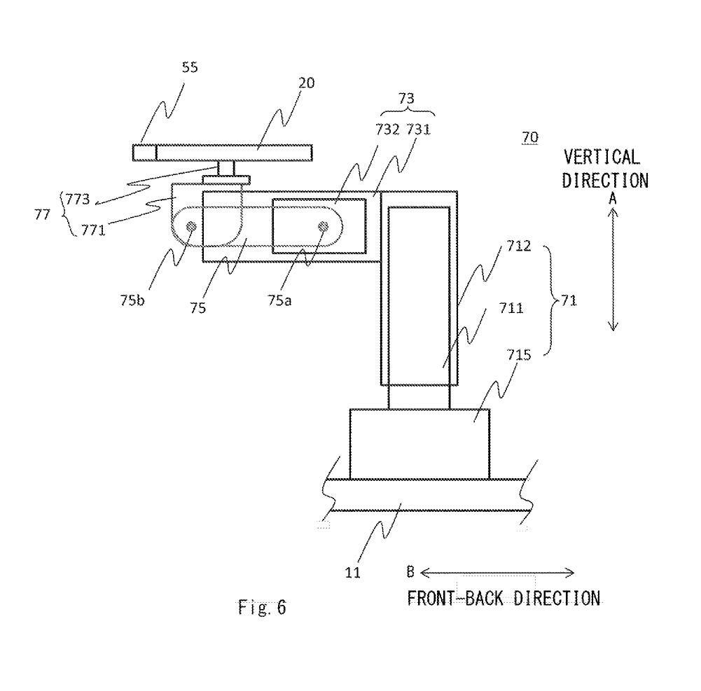

First, the support mechanism of the operation panel 20 will be described with reference to FIG. 6. FIG. 6 is a schematic side view illustrating the mechanism when seen on a lateral side of the ultrasound diagnostic device 100. In the figure, arrow A represents a vertical direction of the device, and arrow B represents a front-back direction of the device. As shown in the figure, the support mechanism 70 includes a vertical moving part 71 that is mounted to a bottom surface of the housing 11 to be slidable in the front-back direction, a horizontal moving part 73 that is mounted to an upper end of the vertical moving part 71 to be movable in the front-back direction, a swing part 75 that is mounted to the horizontal moving part 73 at one end thereof to be rotatable around a shaft 75a, and a rotating part 77 that is mounted to the other end of the swing part 75 to be rotatable around a shaft 75b. The rotating part 77 includes a rotating shaft supporter 771 that is rotatably mounted to the swing part 75 and a rotating shaft 773, and the operation panel 20 is fixed to a plate member (not shown) fixed to the rotating shaft 773 of the rotating part 77 by a fastening screw or the like.

The vertical moving part 71 includes a fixing member 715 that slides on a front-back rail (not shown) fixed to the bottom surface of the housing 11, a support column 711 fixed to the fixing member 715, and a cylinder 712 that is fitted to the support column 711 and moves in the vertical direction along the support column 711. The horizontal moving part 73 is fixed to an upper end of the cylinder 712. The support column 711 and the cylinder 712 may be configured by a known drive mechanism such as a hydraulic cylinder or an air cylinder, and may be operated by an operating pedal or a handle (not shown).

The horizontal moving part 73 includes a guide 731 fixed to the upper portion of the cylinder 712, and a slider 732 that is slidably engaged with the guide 731. An end of a swing arm which forms the swing part 75 is fixed to one end of the slider 732. The rotating shaft supporter 771 of the rotating part 77 is fixed to the other end of the swing arm 75.

Due to the support mechanism 70, the operation panel 20 fixed to the rotating part 77 can perform rotation around the shaft 773, a front-back movement, and a vertical movement, and can perform a vertical movement with a front-back movement according to change in an angle of the swing arm 75 with respect to the horizontal direction. By combining the operations of these mechanisms, it is possible to achieve various movements of the operation panel 20.

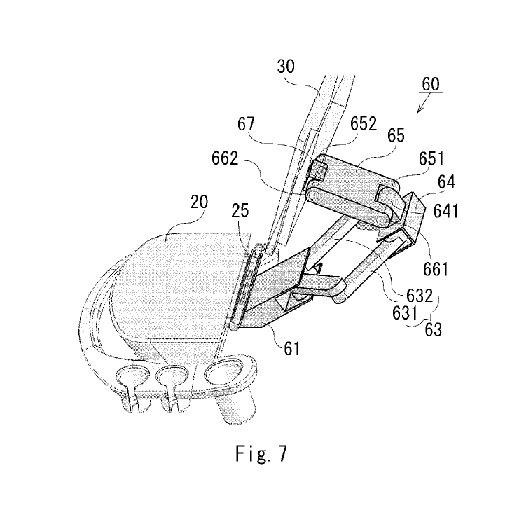

Next, details of the supporter (connecting mechanism 60) of the main display 30 will be described with reference to FIGS. 7 to 12.

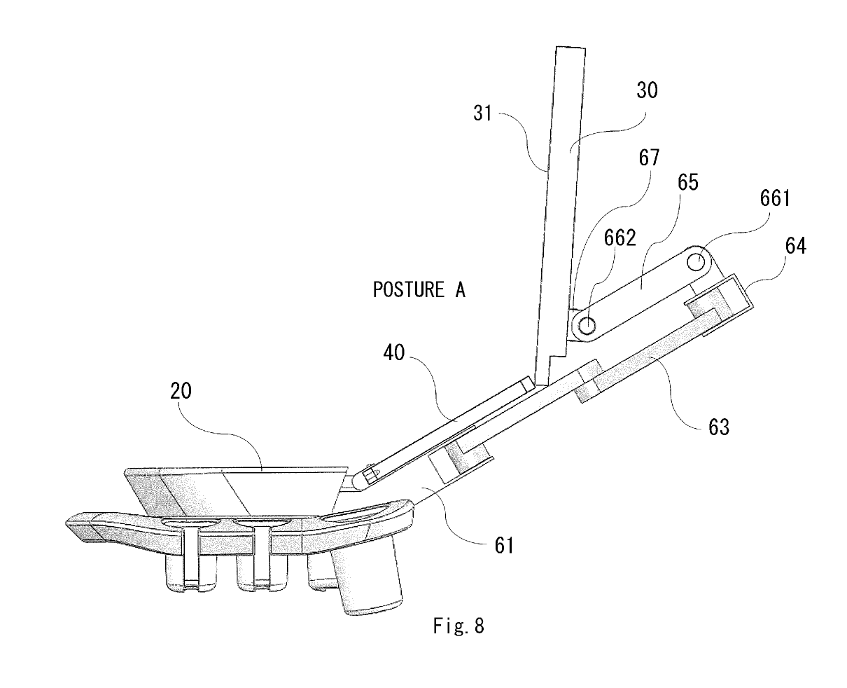

As shown in FIGS. 7 and 8, the connecting mechanism 60 includes a fixing part 61 fixed to the upper surface of the housing 11 of the main body 10, a pantograph-shaped link part 63 that is connected to the fixing part 61 at one end thereof, and a rotating part 65 that connects the other end of the link part 63 to the rear surface of the main display 30, as main components.

The fixing part 61 is a member having a structure in which two plate portions (upper plate and lower plate) are connected to each other at a predetermined interval. One end of the link part 63 is fixed between the two plate portions to be rotatable around an axis that extends in a direction orthogonal to a plate surface of the plate portions. The fixing part 61 is fixed to the upper surface of the housing 11 of the main body 10 by a fastening screw or the like so that the plate portions are inclined with respect to the panel surface 21 of the operation panel 20.

The link part 63 is a link having a pantograph structure in which a pair of right and left link arms 631 and 632, each of which has two arms connected to each other, are combined. One end of each of the link arms 631 and 632 is pivotally supported to the fixing part 61, and the other end thereof disposed at a diagonal position with respect to the one end is fixed to an intermediate member 64 having the same structure as that of the fixing part 61. A distance between the fixing part 61 and the intermediate member 64 is changed according to an opening and closing operation of the pair of link arms 631 and 632 which are disposed between the fixing part 61 and the intermediate member 64, and thus, a distance between the main display 30 and the operation panel 20 that are connected to the intermediate member 64 through the rotating part 65 may be changed. That is, the link part 63 functions as an expansion/contraction mechanism.

The intermediate member 64 is formed by a plate member having a channel-shaped (U-shaped) section, and the rotating part 65 is rotatably fixed to an upper plate portion thereof.

The rotating part 65 is a rectangular parallelepiped member having a small thickness with respect to sizes of a length and a width thereof, and on two opposite side surfaces thereof, shaft supporters 651 and 652 that fixes shafts which are parallel to the side surfaces are formed. A shaft 661 supported by one shaft supporter 651 passes through a shaft supporter 641 fixed to the intermediate member 64, and thus, the rotating part 65 is rotatable around the shaft 661 with respect to the intermediate member 64. A shaft 662 supported by the other shaft supporter 652 passes through a shaft supporter 67 fixed to the rear surface (surface opposite to the display surface 31) of the main display 30, and thus, the main display 30 is rotatable around the shaft 662 with respect to the rotating part 65.

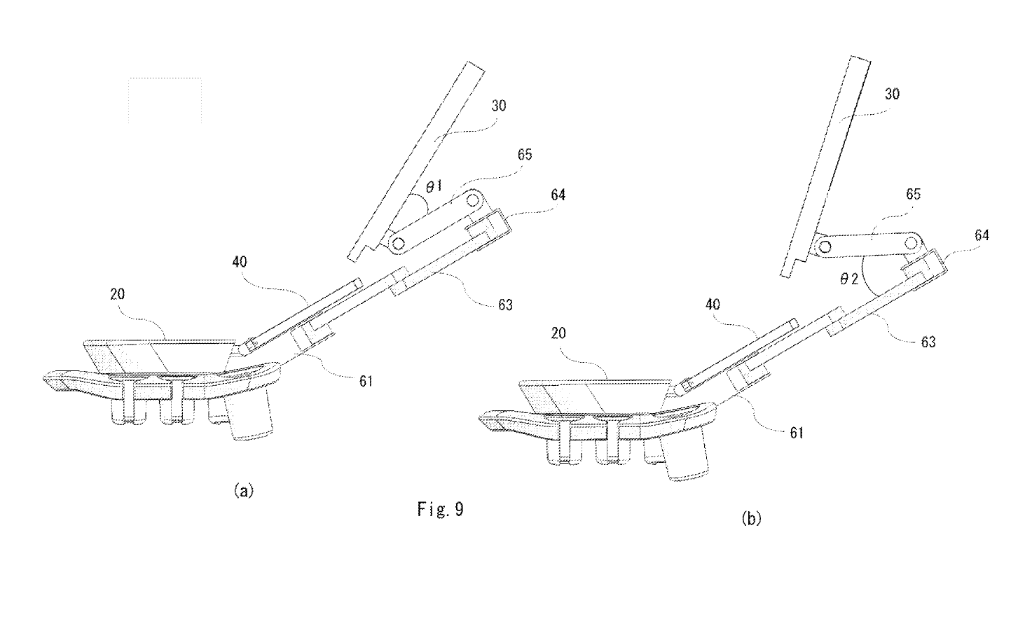

The main display 30 is disposed at a rear and upper position with reference to the sub display 40 connected to the operation panel 20 at a posture A of the sub display 40 shown in FIG. 8, by the connecting mechanism 60 with such a configuration. As shown in FIG. 9(a), by changing an angle θ1 of the main display 30 with respect to the rotating part 65 from the above position, it is possible to change the angle of the display surface 31 into an angle for easy viewing of an operator. Further, as shown in FIG. 9(b), by changing an angle θ2 of the rotating part 65 with respect to the link part 63, it is possible to change a vertical position of the main display 30.

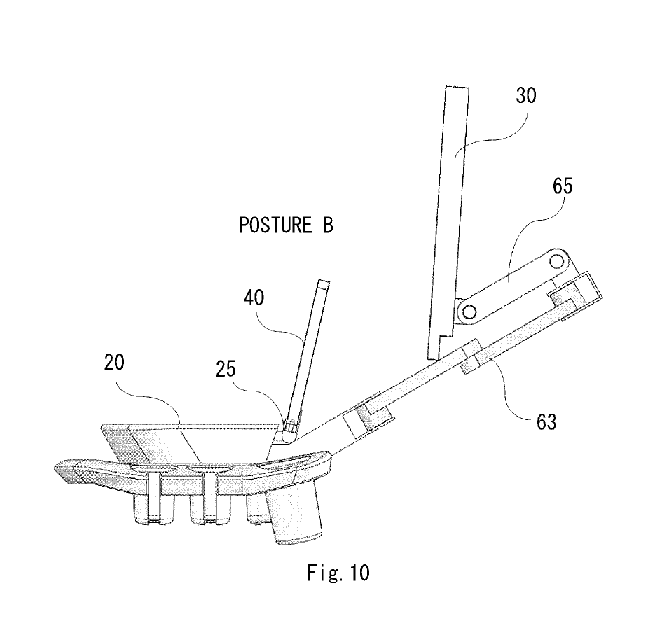

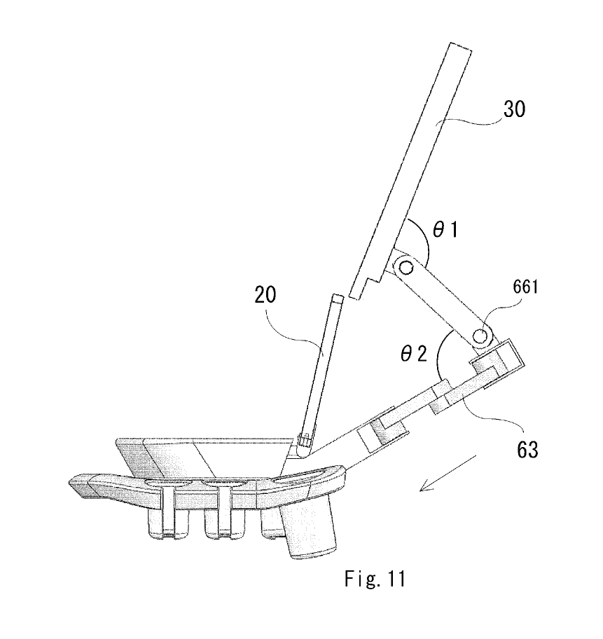

Further, as shown in FIG. 10, by changing the angle of the connection unit 25 with respect to the operation panel 20, when the sub display 40 is disposed at an angle (posture B) close to a vertical posture compared with the posture shown in FIG. 8, for example, as shown in FIG. 11, by increasing the angle θ1 of the main display 30 with respect to the rotating part 65 or the angle θ2 of the rotating part 65 with respect to the link part 63 and changing a distance between one pair of link arms of the link part 63, it is possible to move the main display 30 to be close to the sub display 40 as indicated by an arrow, and thus, it is possible to position the main display 30 on an upper side of the sub display 40.

Here, when the main display 30 moves to be close to the sub display 40 and then rotates, the main display 30 collides with the sub display 40. This collision can be prevented if the operator surely performs the change of the angle of the main display 30 prior to the operation of the link part 63, but the operation of the link part 63 may be mechanically restricted in association with the position of the main display 30. Such a mechanism may employ various locking mechanisms.

For example, when a locking mechanism of the link part 63 that interlocks with the operation of the rotating part 65 is provided, and when the angle θ2 with respect to the link part 63 is small, that is, in a state where the rotating part 65 is folded with respect to the link part 63, the locking mechanism locks the operation of the link part 63 to prevent the main display 30 from moving to the sub display 40. Further, the rotating part 65 rotates to reach a predetermined angle, the locking may be released. In this way, by restricting an expansion/contraction range of the link part 63 in association with the rotation angle of the rotating part 65, it is possible to avoid collision of the main display 30 and the sub display 40 regardless of the opening angle of the sub display 40, and to achieve various postures of the sub display 40.

As another method, a locking mechanism may be provided in the connection unit 25 of the sub display 40. Specifically, a configuration in which a wire wound by rotation (rotation in a clockwise direction shown in FIG. 9) around the shaft 661 of the rotating part 65 is connected between the shaft 661 and the main body side to which the link part 63 is fixed and locking and releasing between the wire and the main body side are performed by the locking mechanism of the connection unit 25 may be used. When the sub display 40 is connected to the connection unit 25, the wire is connected to the main body side, and when the rotating part 65 rotates to lift the main display 30 upward, the link part 63 is operated to be pulled by the wire, so that the main display 30 moves close to the main body side. That is, the main display 30 moves forwardly at a high position such that the main display 30 does not collide with the sub display 40 connected to the connection unit 25. Further, when the sub display 40 is detached from the connection unit 25, the locking mechanism provided in the connection unit 25 is released, and the connection between the wire and the main body side is released. Thus, it is possible to independently perform movement of the main display 30 by means of the link part 63 and movement of the main display 30 by means of the rotating part 65, respectively.

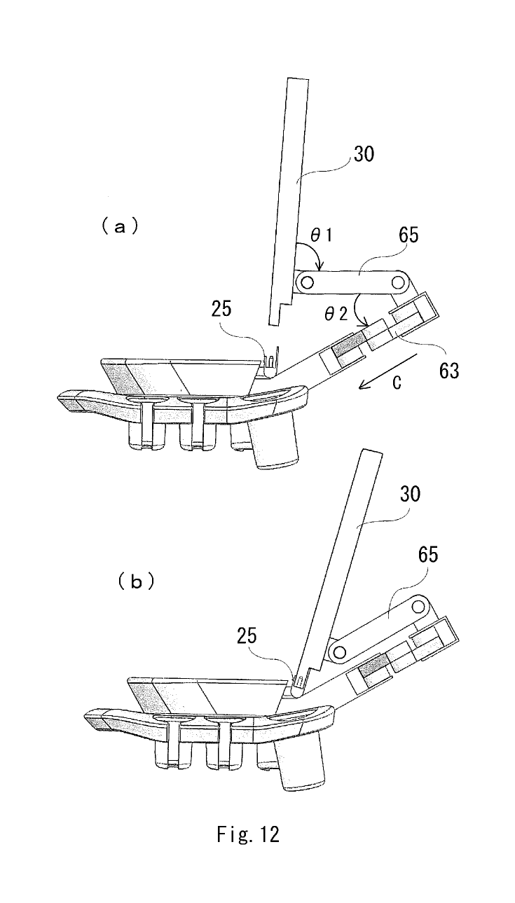

Further, in a state where the sub display 40 is detached from the connection unit 25 of the operation panel 20, as shown in FIGS. 12(a) and 12(b), the main display 30 moves forwardly by the link part 63 (arrow C), and the angle θ2 of the rotating part 65 with respect to the link part 63 and the angle θ1 of the main display 30 with respect to the rotating part 65 are changed, and thus, it is possible to connect the main display 30 to the connection unit 25.

As described above, by operating the link part 63 and the rotating part 65, the main display 30 can move from the rear position of the sub display 40 to the upper or front position thereof without collision even in a state where the sub display 40 is connected to the connection unit 25, and can be connected to the connection unit 25 fixed to the operation panel 20.

In the embodiment shown in FIG. 12, the connecting mechanism 60 is provided so that the main display 30 is connected to the connection unit 25 at a position where the link part 63 is contracted (position where a distance between the link arm 631 and the link arm 632 is maximum) and at a position where the angle θ2 of the rotating part 65 with respect to the link part 63 is minimum, but the sizes of the respective members that form the connecting mechanism 60 are not limited to the embodiment shown in the figures. The sizes may be changed so that the operation range of the main display 30 is enlarged.

For example, when the size of the link part 63 is designed so that the distance between the link arms can be increased from the state where the main display 30 is connected to the connection unit 25, even in a state where the main display 30 is connected to the connection unit, it is possible to change the angle of the main display 30 with respect to the operation panel 20 so that the main display 30 is forwardly inclined compared with the position shown in FIG. 12. Further, the size of the rotating part 65 and the connecting position thereof with the main display 30 may be designed so that the main display 30 is connected to the connection unit 25 at a position where the angle θ2 of the rotating part 65 is larger than 0°, and thus, it is possible to change the angle of the main display 30 so that the main display 30 is backwardly inclined compared with the position shown in FIG. 12.

Hereinbefore, the mechanism of the supporter (connecting mechanism 60) of the main display 30 is described with reference to FIGS. 7 to 12, but one characteristic of the ultrasound diagnostic device of this embodiment is in that the main display 30 which is connected to the main body 10 and the detachable sub display 40 can be connected to the common connection unit 25. Accordingly, the supporter of the main display 30 (connecting mechanism 60) is not limited to the above-described supporter, and any mechanism may be employed as long as the mechanism is capable of moving the end portion of the main display 30 to a position where the end portion is connected to the connection unit 25 provided in the main body 10. For example, instead of the pantograph-shaped link part 63 which is a mechanism capable of expanding or contracting the distance between the connection unit 25 and the main display 30, a link mechanism formed by only one link arm, a slide mechanism formed by a cylinder and a slider, or the like may be employed.

According to the ultrasound diagnostic device of this embodiment, since the detachable sub display 40 is provided separately from the main display 30 supported by the main body, when the sub display 40 is connected to the main body, it is possible to enlarge an effective display area of the display panel. Further, it is possible to realize various display forms, for example, to display plural images having different times and modes to be easily viewed, or to use one display for image display and the other display for a GUI, for example. Further, when the sub display 40 is detached from the main body, it is possible to move the sub display 40 to a free position, and thus, it is possible to allow a patient who lies on a bed or other persons to also view an image at an easy viewing position.

Further, according to the ultrasound diagnostic device of this embodiment, since the connection unit on the main body side to which the detachable sub display 40 is connected also functions as the connection unit for the main display 30, when the sub display 40 is detached from the main body, it is possible to connect the main display 30 to the connection unit, and to perform display at a stable posture.

### Second Embodiment

An ultrasound diagnostic device of a second embodiment is characterized in that a turning function of a display panel supported by a main body is additionally provided.

That is, in the ultrasound diagnostic device of this embodiment, the supporter of the first display includes the rotating part fixed to the rear surface of the display panel, the expansion/contraction mechanism part that is fixed to the main body side at one end thereof and has the variable distance between the main body and the other end thereof, and the intermediate member that connects the rotating part to the expansion/contraction mechanism part. Here, the rotating part is fixed to the rear surface of the display surface through a pivot part fixed to the rear surface, and the display panel is supported to the rotating part in a turnable manner (Aspect 1.

Alternatively, the ultrasound diagnostic device of this embodiment has a characteristic that the supporter of the first display supported by the main body has a function of turning the first display. That is, the ultrasound diagnostic device of this embodiment has a configuration in which the supporter of the first display includes the rotating part that is rotatably fixed to the rear surface of the display surface, the expansion/contraction mechanism part that is fixed to the main body side at one end thereof and has the variable distance between the main body and the other end thereof, and the intermediate member that connects the rotating part to the expansion/contraction mechanism part. The rotating part is connected to the intermediate member in a turnable manner (Aspect 2).

Anyone of the mechanism (pivot) (Aspect 1) that connects the display to the rotating part in a turnable manner and the mechanism (Aspect 2) that connects the rotating part to the intermediate member in a turnable manner may be provided, or both of them may be provided.

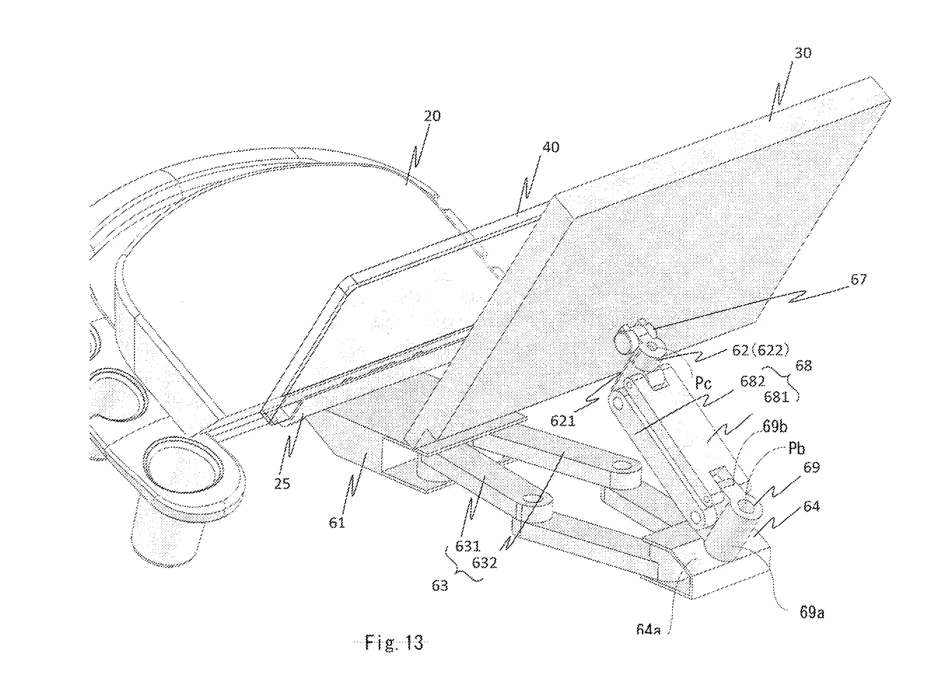

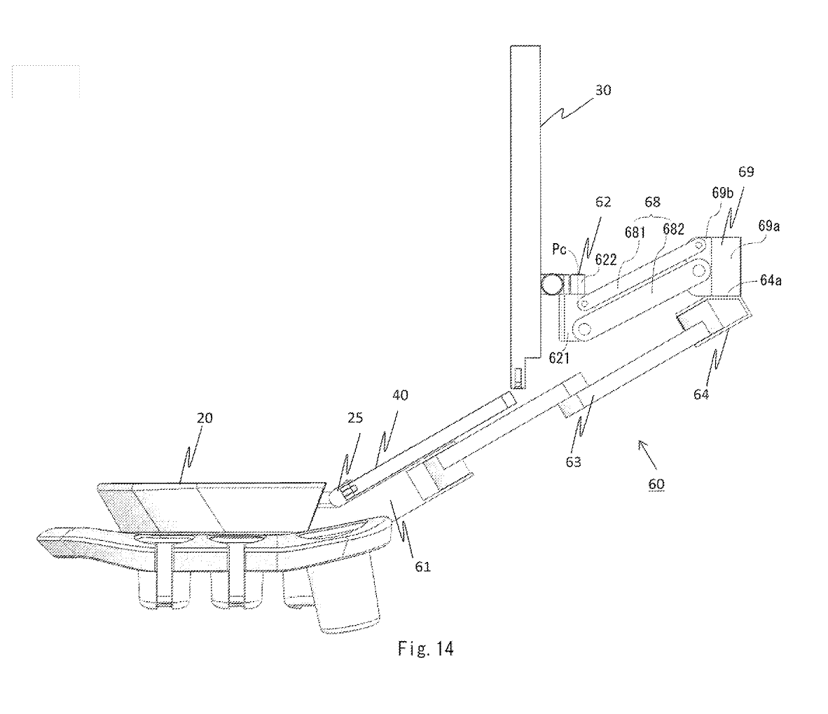

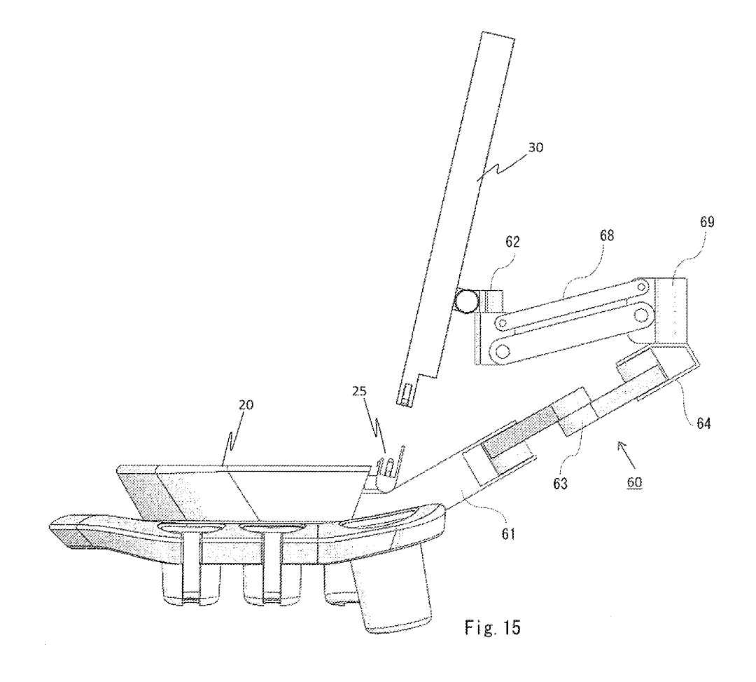

In the ultrasound diagnostic device of this embodiment, since configurations other than the supporter of the first display are the same as in the first embodiment, description thereof will not be repeated, and hereinafter, different mechanisms will be described with reference to FIGS. 13 to 15. FIG. 13 is a perspective view illustrating a support mechanism seen on a rear side of the device, FIGS. 14 and 15 are side views thereof. In FIGS. 13 to 15, the same reference numerals are given to the same components as in the first embodiment, and description thereof will not be repeated.

A supporter 60 of a first display of this embodiment includes a link part 63, an intermediate member 64, a second link part 68, a shaft member 69 fixed to an intermediate member 64, and a pivot part 62 fixed to a rear surface 30b of a main display 30.

The link part 63 includes a pair of link arms, similar to the first embodiment shown in FIG. 1. One end of each link arm is rotatably shaft-supported to a fixing part 61 fixed to a main body 10, and the other end thereof is rotatably shaft-supported to the intermediate member 64. The intermediate member 64 is a member having a structure in which shafts of the link arms are inserted between two parallel plates similar to the first embodiment, in which an upper plate portion has an inclined surface 64a formed on a rear side with reference to a portion thereof where the shafts of the link arms are supported between the upper plate portion and a lower plate portion. The shaft member 69 is fixed to the inclined surface 64a. That is, as shown in the side view of FIG. 14, the plate portions of the intermediate member 64 that shaft-supports the link part 63 are disposed on a flat plane parallel to the link part 63 fixed at an angle with respect to an operating surface of an operation panel 20, but the inclined surface 64a that is inclined with respect to the plate portions is disposed on an approximately horizontal plane, and thus, vertically supports the shaft member 69.

The shaft member 69 is a member including a cylindrical member 69a and a plate-shaped member 69b that extends in a radius direction of the cylindrical member, in which the cylindrical member 69a is fitted to a shaft Pb provided on the upper plate portion of the intermediate member 64, and the plate-shaped member 69b shaft-supports an end portion of the second link part 68. Thus, the shaft member 69 can rotate around the shaft Pb provided in the intermediate member 64, so that the second link part 68 connected to the plate-shaped member 69b of the shaft member 69 and the main display 30 can be turned around the shaft. Here, a configuration in which the shaft member 69 is rotatably supported to the intermediate member 64 is described, but a configuration in which the shaft member 69 is fixed to the intermediate member 64 and does not turn is included in this embodiment.

The second link part 68 includes a pair of arms 681 and 682 which are vertically disposed, and a concave portion for being engaged with the shaft member 69 and the pivot part 62 is formed on opposite end portions of each of the arms 681 and 682. At the end portion connected to the shaft member 69, the plate-shaped member 69b of the shaft member 69 is fitted to the concave portions of the upper and lower arms 681 and 682, so that the upper and lower arms can rotate with respect to the shaft member 69.

The pivot part 62 has a structure in which two members of a supporter 621 having a structure similar to that of the shaft member 69 and a turning part 622 that is rotatably connected to the supporter 621 are combined. The supporter 621 and the turning part 622 are members having a cylindrical portion and a plate-shaped portion that extends in a radius direction of the cylindrical portion. Concave portions formed in end portions of the upper and lower arms 681 and 682 are fitted to the plate-shaped portion of the supporter 621 to support the arms 681 and 682 to be rotatable. Further, a pivot Pc is fixed to the cylindrical portion of the supporter 621 along its central axis, the cylindrical portion of the turning part 622 is fitted to the pivot Pc, so that the turning part 622 can rotate around the pivot Pc. The plate-shaped portion of the turning part 622 is shaft-supported to the shaft supporter 67 fixed to the rear surface of the main display 30, so that the main display 30 can rotate with respect to the supporter 621.

In such a configuration of the supporter 60, a configuration in which the distance between the main display 30 and the operation panel 20 is expanded or contracted using the link part 63, and a configuration in which an angle of the second link part 68 with respect to the link part 63 or an angle of the main display 30 with respect to the second link part 68 is changed to change an angle of the display panel with respect to the vertical direction or a position thereof in the vertical direction are the same as in the first embodiment. Further, as shown in FIG. 15, a configuration in which the main display 30 can be connected to the connection unit 25 by combining respective rotations of the link part 63 and the second link part 68, in a state where the detachable sub display 40 is not connected to the display connection unit 25 provided in the operation panel 20, is also the same as in the first embodiment.

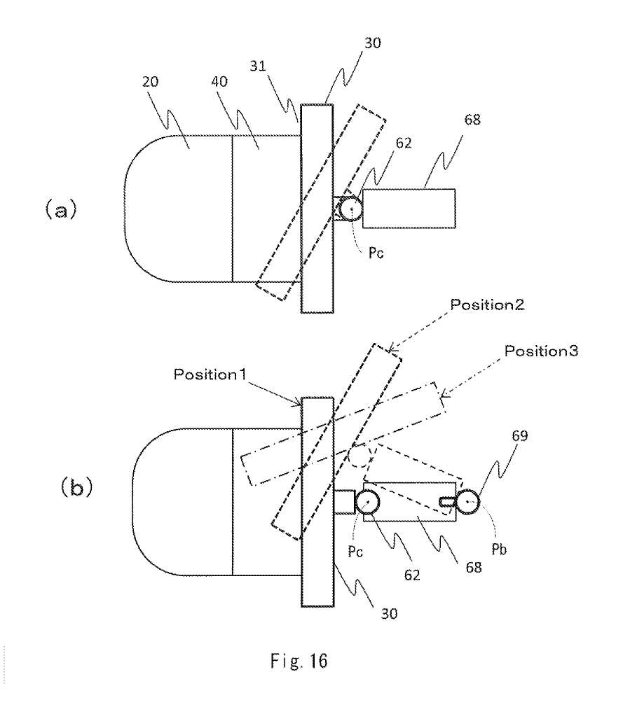

In addition to these operations, in the support mechanism of this embodiment, the main display 30 may turn around the shaft member 69 and/or the pivot part 62 (Pc). For example, as shown in FIG. 16(a), the main display 30 may turn around the pivot part 62 to change the direction of the display surface 31. Further, as shown in FIG. 16(b), the second link part 68 and the main display 30 may turn around the shaft member 69 to move the main display 30 from an initial position (Position 1) to a turning position (Position 2), and to further turn the main display 30 from the position (Position 2) around the pivot part 62 (turning to Position 3).

In a state where the main display 30 is disposed on a rear side of the sub display 40 as shown in FIG. 14, since a turning range of the main display 30 does not overlap a movement range where the sub display 40 is opened or closed with respect to the operation panel 20, it is possible to reliably prevent collision between them. Further, as described in the first embodiment, by providing a restriction based on the angle of the second link part 68 to the movement of the main display 30 produced by the link part 63, or by providing a locking mechanism that is operated according to the presence or absence of connection of the sub display 40 to the connection unit 25, it is possible to perform a turning operation of the main display 30 while preventing collision with the sub display 40 not only at the position shown in FIG. 14 but also in various opening or closing angles of the sub display 40.

As described above, according to the ultrasound diagnostic device of this embodiment, it is possible to provide various movements including turning of the main display 30, in addition to the effects of the first embodiment.

In this embodiment, similar to the first embodiment, instead of the pantograph-shaped link part 63, as a mechanism capable of expanding or contracting the distance between the main display 30 and the connector 25, a link mechanism configured by only one link arm, a slide mechanism configured by a cylinder and a slider, or the like may be employed.

### Modification Example

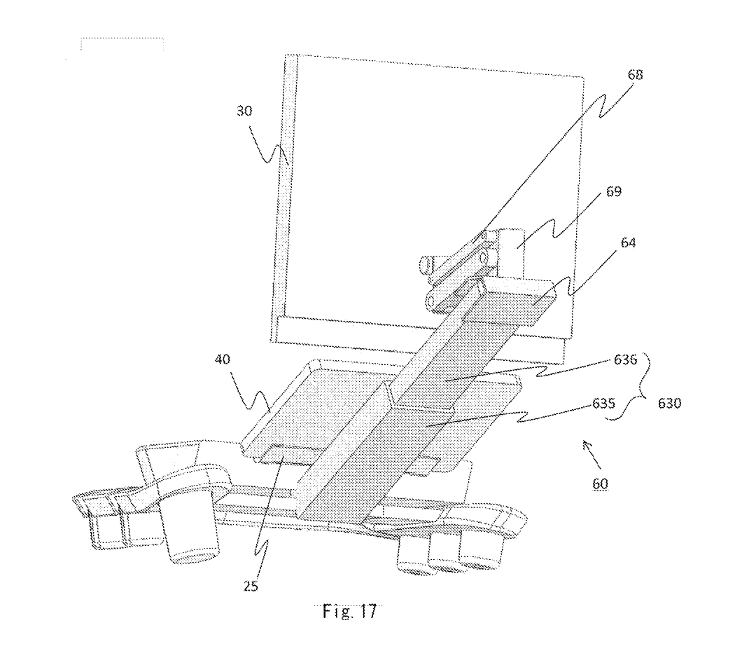

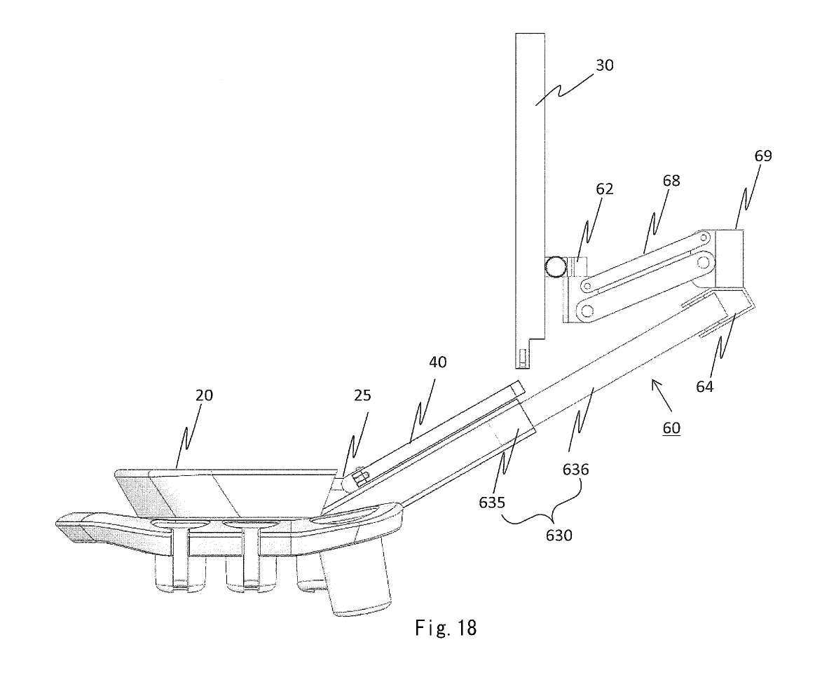

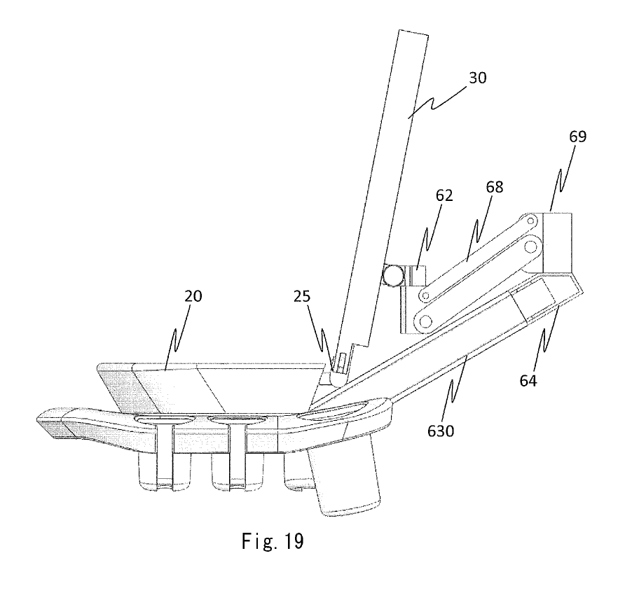

Instead of the link part 63 of the second embodiment, a modification example of the supporter (connecting mechanism) 60 that employs a slide mechanism are shown in FIGS. 17 to 19. FIG. 17 is a perspective view illustrating the supporter 60 when seen on a rear side of the device, FIG. 18 is a side view illustrating a state where the sub display 40 is connected to the display connection unit 25, and FIG. 19 is a side view illustrating a state where the sub display 40 is detached from the display connection unit 25. In the figures, the same reference numerals are given to the same components as in the first and second embodiments.

As shown in the figures, the supporter 60 of a display employed in an ultrasound diagnostic device of this modification example employs a slide mechanism 630 as a mechanism that moves the main display 30 in a front-back direction. The slide mechanism 630 includes a hollow cylindrical body 635 and a slider 636 that slides inside the cylindrical body, in which the cylindrical body is fixed to a main body side at a predetermined angle, and the slider is fixed to the intermediate member 64 at an end portion thereof disposed outside the cylindrical body. A support structure from the intermediate member 64 to the main display 30 is the same as in the second embodiment.

In this modification example, the slider 636 slides so that the main display 30 can move in the front-back direction, from a position in back of the sub display 40 as shown in FIG. 18 to a front position where the main display 30 is connected to the connection unit 25 as shown in FIG. 19. In this movement, a configuration in which the main display 30 moves in the vertical direction using rotation produced by the second link part 68 and a configuration in which the main display 30 is turned using rotation produced by the shaft member 69 or the pivot part 62 are the same as in the second embodiment.

### Third Embodiment

An ultrasound diagnostic device of a third embodiment is characterized in that a slide mechanism is additionally provided to a connector of a display. Since other configurations are the same as in the first and second embodiments, different points will be mainly described hereinbefore.

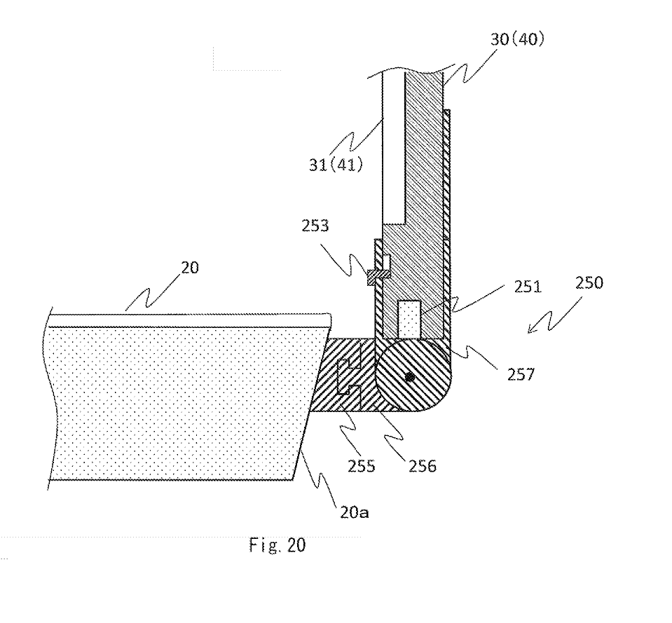

FIG. 20 is a sectional view illustrating a connection unit 250 fixed to the operation panel 20. The connection unit 250 includes a fixing part 255 fixed along an end surface 20a of the operation panel 20, a sliding part 256 that is connected to the fixing part 255 and is movable along the end surface 20a of the operation panel 20, and a display accommodating part 257 that is rotatably mounted to the sliding part 256.

Similar to the connection unit 25 of the first embodiment shown in FIGS. 4 and 5, the display panel accommodating part 257 is an elongated member formed by a concave portion having a U-shaped section, and is mounted to the sliding part 256 so that its length direction is parallel to the rear surface of the operation panel 20. Thus, angles of the displays 30 and 40 connected to the display panel accommodating part 257 with respect to the operation panel 20 can be changed. A configuration in which a connector 251 for electrically connecting the display 30 or 40 to the main body 10 and a protrusion 252 which serves as a guide when the display is inserted into the accommodating part are provided in the concave portion of the display accommodating part 257, and a configuration in which a locking mechanism 253 that fixes the display is provided, are the same as in the first embodiment.

An end surface of the fixing part 255 to which the sliding part 256 is connected is formed in a rail shape, and a key-shaped end surface of the sliding part 256 is engaged with the rail-shaped end surface so that both of the end surfaces are connected to each other, and thus, the sliding part 256 can slide.

In this way, since the connection unit 250 has the slide mechanism, movement of the display in the lateral direction, in addition to the opening or closing operation of the display with respect to the operation panel 20, can be performed, and thus, it is possible to move the display panel connected to the connection unit 250 to a position for easy viewing. Further, when the sub display 40 is connected to the connection unit 250, the sub display 40 is at a position parallel to the main display 30 which is the other display, and the main display 30 and the sub display 40 do not collide with each other due to the movement of the connection unit 250 in the lateral direction.

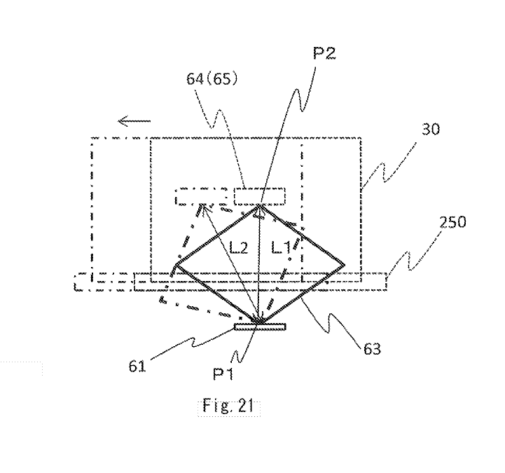

When the main display 30 is connected to the connection unit 250, the end portion of the main display 30 connected to the connection unit 250 and the end portion (fixing part 61) of the supporter 60 fixed to the main body 10 cause lateral deviation when the connection unit 250 slides, but it is possible to absorb the deviation by expansion/contraction and rotation of the link part 63 of the supporter 60. This state is shown in FIG. 21.

In FIG. 21, a point P1 represents a position where one end of the link part 63 is shaft-supported by the fixing part 61 fixed to the main body side. A point P2 represents a position where the other end of the link part 63 is shaft-supported by the intermediate member 64 fixed to the main display 30 through the shaft supporter 67 and the rotating part 65. When the main display 30 moves from a position (position A) indicated by a dashed line to a position (position B) indicated by a single dot chain line as the connection unit 250 moves in a direction indicated by an arrow in the figure, the point P1 of the link part 63 is stationary, but the point P2 linearly moves in the horizontal direction according to the movement of the main display 30. Here, as the link part 63a rotates around the point P1 while making a distance L1 between the point P1 and the point P2 lengthened (represented as L2, in which L2>L1) compared with a distance in a case where the display 30 is disposed at the position A, the link part 63 thus follows the horizontal movement of the intermediate member 64 and the rotating part 65, that is, the horizontal movement of the main display 30. In this way, even when the lateral deviation is present between the position where the main display 30 is fixed to the main body side through the supporter 60 and the position where the main display 30 is fixed to the main body side through the connection unit 250, the supporter 60 can ensure a smooth operation of the main display 30 by its connecting mechanism.

According to this embodiment, since it is possible to cause the display connected to the connection unit 250 to slide in the lateral direction of the device, for example, it is possible to move an image on the display to be close to a person who wants to the image, and also to secure a wide space beside the display when moving an ultrasound probe connected to the ultrasound diagnostic device.

### Fourth Embodiment

Next, an embodiment of an operation of the ultrasound diagnostic device of the invention will be described.

As described above, a main characteristic of the ultrasound diagnostic device of the invention is in that the sub display 40 is detachably mounted to the main body 10. This embodiment is characterized in that a simple ultrasound examination function is provided to the detachable display panel so that functions of the ultrasound diagnostic device are enlarged.

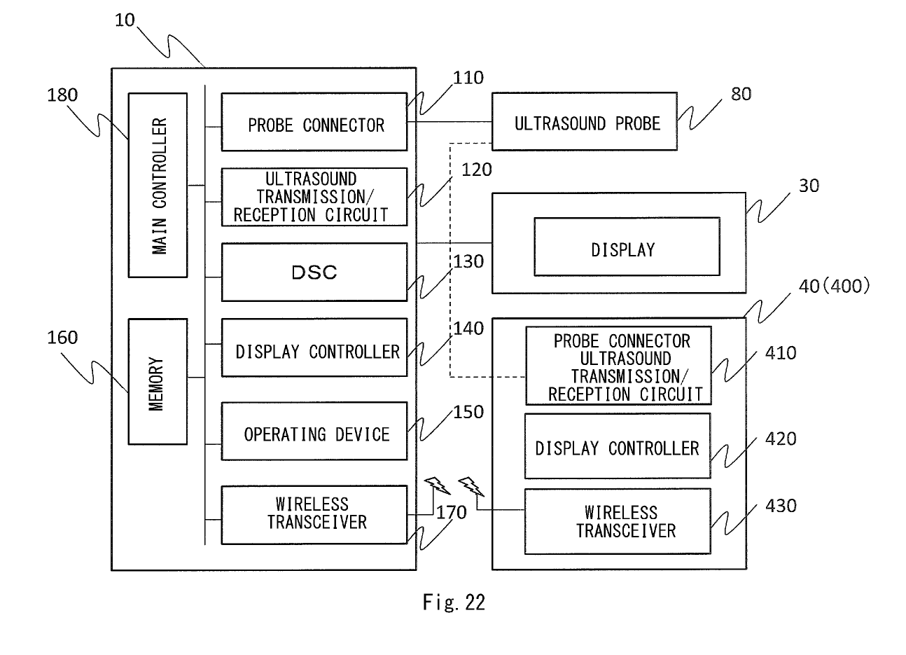

A functional block diagram of the sub display 40 capable of functioning as a simple ultrasound diagnostic device and a functional block diagram of the main body 10 are shown in FIG. 22.

As shown in the figure, the main body 10 includes a probe connector 110 to which an ultrasound probe 80 is connected; a transmission/reception circuit 120 that transmits an ultrasound signal to the ultrasound probe 80 through the probe connector 110 and receives an echo signal (signal obtained by converting ultrasound into an electric signal using a transducer) received by the ultrasound probe 80; a digital scan converter (DSC) 130; a display controller 140 that displays an ultrasound image such as an M mode or a B mode, processed and imaged by the digital scanning converter 130 on the main display 30 and the sub display 40; an operating device 150 (including the operation panel 20) for inputting a condition or a command necessary for examination to the main body 10; a memory 160 that stores parameters or data necessary for examination, an ultrasound image, and the like; a wireless transceiver 170 that performs wireless communication with the sub display 40 detached from the main body 10; and a main controller 180 that controls overall operations of the respective components. A program or data for operating various functions provided in the ultrasound diagnostic device is provided in the main controller 180 and the memory 160. Further, a display (main display 30) is connected to the main body 10.

Although not shown in FIG. 22, an auxiliary device, a power supply, or the like provided in a normal ultrasound diagnostic device may be further provided.

Since the functions of the main body 10 other than the display controller 140 are the same as in a conventional ultrasound diagnostic device, detailed description about respective components will be omitted. The display controller 140 switches a display mode between a state where the sub display 40 is connected to the main body 10 and a state where the sub display 40 is detached from the main body 10. Detailed operation thereof will be described later.

In order to realize a limited ultrasound examination function, the sub display 40 includes a probe connector or an ultrasound transmission/reception circuit 410, a display controller 420, and a wireless transceiver 430. In addition, although not shown, the sub display 40 may further include a controller and a memory. The limited ultrasound examination function refers to a function for performing limited examination with respect to a limited portion necessary for a round visit in a hospital, or the like, and may be provided in the controller or the memory provided in the sub display 40 as a package. Alternatively, the sub display 40 may be connected to the main body 10 to read the necessary function from the main controller 180 or the memory 160 in the main body 10. A mechanism of a connection unit that connects the sub display 40 to the main body 10 may employ the mechanism described in the first to third embodiments.

The ultrasound diagnostic device 100 having such a configuration may be operated in an operating mode (dual-display mode) where the sub display 40 is connected to the main body 10 and an operating mode (mono-display mode) where the sub display 40 is not connected to the main body 10. Further, the ultrasound diagnostic device 100 may be operated in an operating mode (image reading mode) where the sub display 40 that is not connected to the main body 10 is operated as a simple ultrasound diagnostic device 400 and performs transmission or reception with the main body 10 in a wireless manner, and an ultrasound image obtained by the simple ultrasound diagnostic device 400 is displayed on the main display 30 of the ultrasound diagnostic device 100.

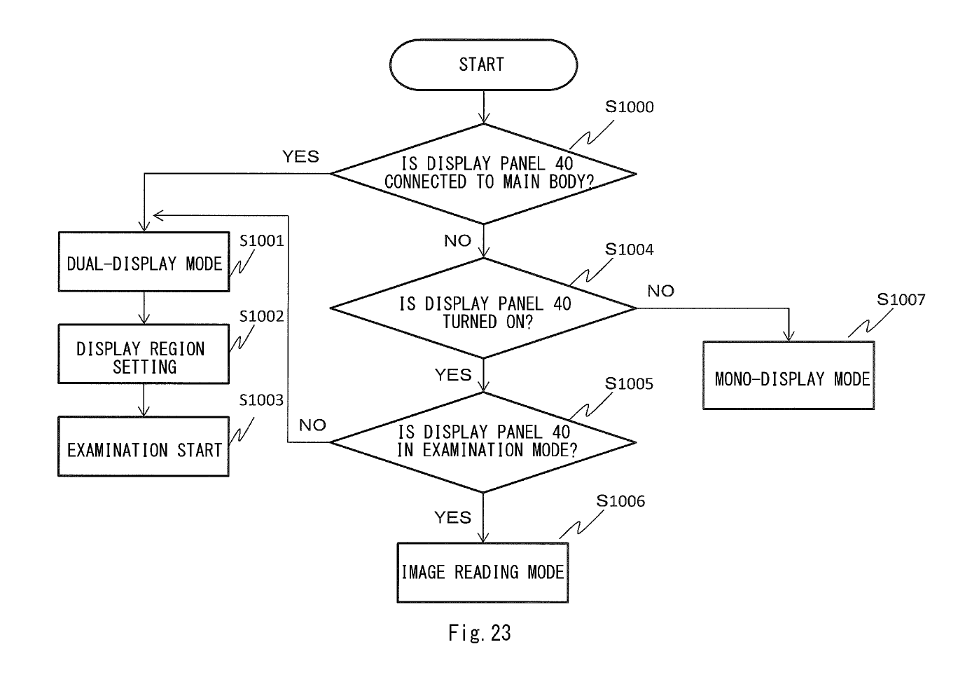

An example of an operation in the ultrasound diagnostic device 100 is shown in FIG. 23.

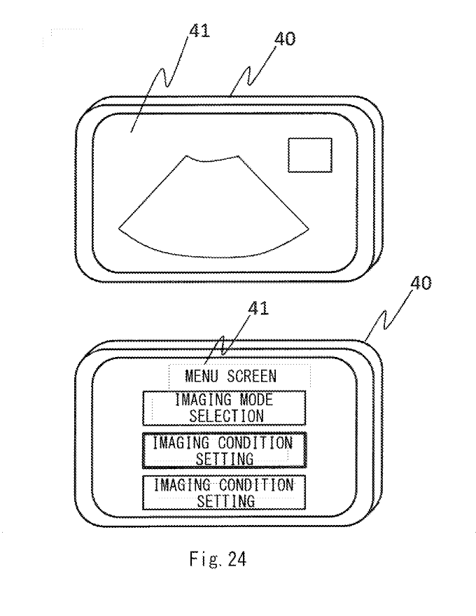

If the ultrasound diagnostic device 100 starts its operation as power is supplied thereto, first, it is determined whether the sub display 40 is electrically connected (S1000). When the sub display 40 is connected, the ultrasound diagnostic device 100 is operated in the dual-display mode where both of the main display 30 that is already connected and the sub display 40 are used as displays (S1001). In this mode, a region for display content of the main display 30 and a region for display content of the sub display 40 are appropriately divided (S1002), and then, examination is started (S1003). The region division is arbitrary, but for example, when the ultrasound diagnostic device having the structure shown in FIG. 1 is used, the sub display 40 that is close to the operation panel 20 may be used as a display for a GUI or the like, and the main display 30 having a large screen may be used as a display for an ultrasound image. Further, images for different sections or images obtained at different times may be displayed on the two display panels, so that an examiner may compare the images. FIG. 24 shows a display example of the display surface 41 of the sub display 40. In FIG. 24, an upper diagram shows a state where an image is displayed, and a lower diagram shows a state where a UI is displayed.

When the sub display 40 is not connected to the ultrasound diagnostic device 100 or when the sub display 40 is detached from the main body from the dual mode state (NO in S1000), it is determined whether the sub display 40 is supplied with power to be operable (S1004), and when the sub display 40 is in the operable state, it is determined whether the operating mode of the sub display 40 is an examination mode or a display mode (S1005).

When the operating mode of the sub display 40 is the examination mode, an echo signal received by the ultrasound transmission/reception circuit 410 of the display panel through the ultrasound probe 80 connected to the sub display 40 is transmitted to the ultrasound diagnostic device 100 by the wireless transceivers 430 and 170. The ultrasound diagnostic device 100 processes the signal in the same way as in a signal received by the ultrasound transceiver 120, and displays the result on the main display 30 by the DSC 130. That is, the ultrasound diagnostic device 100 is operated in the image reading mode where an ultrasound image received by the sub display 40 that functions as the simple ultrasound diagnostic device 400 is displayed on the main display 30 (S1006).

When the sub display 40 is operated in the display mode (NO in S1005), the sub display 40 may be used as an auxiliary display device in the dual-display mode (S1001), or may be used as a touch panel that displays an ultrasound image or receives an input of an examiner.

When power is not supplied to the sub display 40 (NO in S1004), the ultrasound diagnostic device 100 may be operated, in a similar way to a conventional mono-display device (S1007).

According to the ultrasound diagnostic device of this embodiment, it is possible to variously use the sub display 40 according to the mounted or detached state of the sub display 40 which is detachably mounted to the ultrasound diagnostic device 100, and to realize various display modes.

The operation shown in FIG. 23 is an example of an operation of the ultrasound diagnostic device of this embodiment, and the invention is not limited to the shown flow, and modifications may be made. For example, it is preferable that the sub display 40 has a simple examination function in addition to the function of the display panel, but this configuration is not essential, and the sub display 40 may be used as a device only for display. In this case, steps S1005 and S1006 in FIG. 23 are not necessary.

### INDUSTRIAL APPLICABILITY

According to the invention, it is possible to greatly increase the degree of freedom in examination and the degree of freedom in image display, in ultrasound diagnosis.

### REFERENCE SIGNS LIST

10 main body
20 operation panel
25 display connection unit
30 main display (first display)
40 sub display (second display)
50 probe housing
60 supporter (connecting mechanism)
63 link part (expansion/contraction mechanism)
65 rotating part
67 shaft supporter
68 second link part (rotating part)
69 shaft member
70 operation panel support mechanism
80 probe
100 ultrasound diagnostic device
110 probe connector
120 ultrasound transmission/reception circuit
140 display controller
150 operating device
170 wireless transceiver
400 simple ultrasound diagnostic device
420 display controller
430 wireless transceiver

1. An ultrasound diagnostic device comprising:
a main body that includes an operation panel;
a first display that is connected to the main body through a supporter; and
a second display that is detachably connected to the main body,
wherein the second display includes a transmission/reception circuit that performs transmission or reception with the main body.

2. The ultrasound diagnostic device according to claim 1,
wherein the main body includes a display connection unit that is detachably connected to the second display, and the first display and the second display include an end portion that is engaged with the display connection unit.

3. The ultrasound diagnostic device according to claim 1,
wherein the supporter includes a connection mechanism that moves the first display between a first position where the first display is disposed on a rear side of the second display connected to the main body and a second position where the first display is disposed on an upper or front side of the second display.

4. The ultrasound diagnostic device according to claim 3,
wherein the connection mechanism includes an expansion/contraction mechanism part that is fixed to the main body at one end thereof and a rotating part that is connected between the other end of the expansion/contraction mechanism part and the first display, and the rotating part is rotatably connected to each of the expansion/contraction mechanism part and the first display.

5. The ultrasound diagnostic device according to claim 4, further comprising:
a pivot part between the expansion/contraction mechanism part and the rotation part and/or between the rotation part and the first display.

6. The ultrasound diagnostic device according to claim 4,
wherein the expansion/contraction mechanism part includes a link mechanism.

7. The ultrasound diagnostic device according to claim 1,
wherein the second display is a tablet-type display panel including a wireless transmission/reception circuit.

8. The ultrasound diagnostic device according to claim 2,
wherein the display connection unit is provided to be rotatable with respect to the operation panel fixed to the main body.

9. The ultrasound diagnostic device according to claim 2,
wherein the display connection unit is provided to be slidable with respect to the operation panel fixed to the main body.

10. The ultrasound diagnostic device according to claim 1, further comprising:
a controller that operates the ultrasound diagnostic device in any one mode among a dual-display mode where the first display and the second display connected to the main body function together as display panels and a mono-display mode where display content of the second display detached from the main body is displayed on the first display.

11. An ultrasound diagnostic device comprising:
a main body that includes an operation panel;
a first display that is connected to the main body through a supporter; and
a second display that is detachably connected to the main body,
wherein the supporter includes a connection mechanism that moves the first display between a first position where the first display is disposed on a rear side of the second display connected to the main body and a second position where the first display is disposed on an upper or front side of the second display.
